(19) United States
(12) Plant Patent Application Publication
(10) Pub. No.: US 2003/0056269 P1
(43) Pub. Date: Mar. 20, 2003

(54) Chrysanthemum plant named 'Drive Time'

(76) Inventor: Mark Roland Boeder, J H Den Haag (NL)

Correspondence Address: HOWREY SIMON ARNOLD & WHITE, LLP, BOX 34, 301 RAVENSWOOD AVE., MENLO PARK, CA, 94025, US

(21) Appl. No.: 09/957,486

(22) Filed: Sep. 19, 2001

(51) Int. Cl. A01H 5/00

(52) U.S. Cl. PLT/296

(57) ABSTRACT

A new variety of Chrysanthemum plant named ‘Drive Time,’ having medium vigour with an upright and branching habit and slightly domed, semi incurving flower form. The colour is rich orange bronze.

### BACKGROUND OF THE INVENTION

The present invention comprises a new and distinct cultivar of Chrysanthemum botanically known as Dendranthema grandiflora, and referred to by the cultivar name ‘Drive Time’.

‘Drive Time’, identified as 20199-1 originated from a cross made by Mark Boeder in a controlled breeding program in Chichester, West Sussex, United Kingdom. The female parent of the new variety is ‘Peak Time,’ and the male parent is the variety known as ‘Iridion,’ U.S. Plant Pat. No. 6125. The new variety ‘Drive Time’ has been asexually reproduced by vegetative cuttings in Chichester, West Sussex, United Kingdom and the distinguishing characteristics are retained through successive generations of asexual reproduction.

### BRIEF SUMMARY OF THE INVENTION

‘Drive Time’ is a pot type of Chrysanthemum plant variety having a slightly domed capitulum of bright orange bronze decorative flowers.

### Comparison with Parent

Plants of the new Chrysanthemum variety ‘Drive Time’ are similar to the parent cultivar ‘Peak Time’ in plant habitat and growth rate. In side-by-side comparisons in Chichester, West Sussex, United Kingdom, under commercial practice, plants of the new Chrysanthemum variety ‘Drive Time’ differed from plants of the parent cultivar ‘Peak Time’ in the following characteristics.

1. The new variety ‘Drive Time’ produces medium sized orange bronze decorative flowers, whereas the parent cultivar ‘Peak Time’ produces pink decorative flowers.

2. Plants of the new variety ‘Drive Time’ have larger and taller inflorescence than plants of the parent cultivar ‘Peak Time.’

3. ‘Drive Time’ has flowers that are more incurved than ‘Peak Time.’

### Comparison with Other Varieties

Plants of the new Chrysanthemum variety ‘Drive Time’ are dissimilar to the cultivar ‘Orange Bowl’ in plant habitat and growth rate. However, in side-byside comparisons in Chichester, West Sussex, United Kingdom, under commercial practice, plants of the new Chrysanthemum variety ‘Drive Time’ compared to plants of the cultivar ‘Orange Bowl’ in the following characteristics.

1. The new variety ‘Drive Time’ produces orange bronze decorative flowers, whereas the cultivar ‘Orange Bowl’ produces bright orange decorative flowers.

2. Plants of the new variety ‘Drive Time’ have smaller and shorter inflorescence than plants of the cultivar ‘Orange Bowl.’

3. The new variety ‘Drive Time’ has more flowers than ‘Orange Bowl,’ and is faster in response time than ‘Orange Bowl’ by four days.

### BRIEF DESCRIPTION OF ILLUSTRATIONS

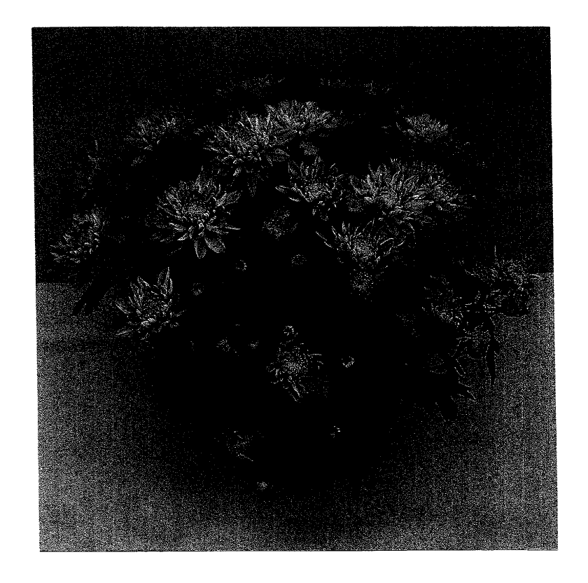

Typical specimens of the plant and flowers for the new Chrysanthemum variety ‘Drive Time’ are shown in the accompanying photograph. The colours shown are as true as possible within the usual limits of this kind of illustration. FIG. 1 is a whole plant view of the new variety ‘Drive Time’ grown in a pot. The plant shown in the illustration is 55 days from start of Short Days.

### DETAILED BOTANICAL DESCRIPTION

The following description of the new Chrysanthemum variety ‘Drive Time’ is of plants grown in a greenhouse in Chichester, West Sussex, United Kingdom in the month of August. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 16° C. at night and about 24° C. to 25° C. during the day under light conditions of about 5,000 to 6,000 foot candles. The plants respond well to the use of growth retardant, such as three B9 treatments at about 2000 ppm. To produce a commercial product the plants may be pinched once with the centre bud removed. The typical container size for commercial growth is 14 cm. It has been observed that the shelf life of the new variety is about 24 days with a response time of about 8 weeks. The new variety is suitable for growth in a temperature range of 16° C. to 25° C.

The new variety may be produced as a spray and disbud. The following description is with respect to a plant produced as Spray pot Chrysanthemums. In the description of this new Chrysanthemum variety, colour values have been taken from The Royal Horticultural Society Colour Chart (R.H.S.C.C.).

### Plant

Plant type: Pot

Habit: Upright and branching

Height: Medium 18 to 20 cm

Width: Near 28 to 30 cm

Branching characteristics: Angular and spreading

Length of Lateral Branches: 10 to 15 cm

Number of Breaks from Pinch: 4 to 5

Stem Colour: 146 B

Response Time: 53 days

Vigour: Medium

Shelf Life: Near 21 days

Disease: No observations

Pest: No Observations

Growth retardant type and treatment: B9 85% 3 applications of 2 gram/litre at 2 days, 21 days and 31 days, after propagating unrooted vegetative cuttings. The plants were grown for two weeks in Long Day conditions (20 hours of light) and then transferred to Short Day conditions (13 hours of dark).

Propagation:

Type.—Vegetative propagation via stem cuttings.

Time to rooting.—10-12 days with soil temperatures of 18° C.

Rooting habit.—Fine and fibrous.

### Foliage

Number of Leaves per Lateral Branch: 10 to 12

Compound or single: Compound

Arrangement of Leaves: Alternate

Shape of leaf.—5 lobed.

Size of leaf.—Width (cm): Near 5 cm Length (cm): Near 7.5 cm

Leaf apex.—Cuspidate.

Base.—Acute.

Attachment.—Petioled.

Aspect.—Slightly undulating.

Margin.—Palmately lobed.

Surface characteristics.—Top: Slightly pubescent and glossy Bottom: Pubescent

Petiole: Colour: Near 137 C Length: 1.5 cm

Venation: Net, prominent central vein and two lobes either side top and underside. Colour: upper side: Near 138 A under side: Near 138 B

Colour: Mature Leaf, upper side: Near 137 A; underside: Near 138 B Young Leaf, upper side: Near 139 A; under side: Near 137 C

### Flower

Flower Appearance: Flat and circular, Ray florets slightly protruding.

Flower type: Decorative

Flower Form: Slightly domed

Flower shape: Circular

Flower Habit: Upright

Number of blossoms per branch: 4 to 5

Inflorescence form: Coryrnbiform

Depth of fully expanded blossoms.—Near 2½ cm.

Diameter of fully expanded blossoms.—Near 5 to 6 cm.

Sepals:

Number.—Approx. 20.

Colour.—Near 137 C.

Length.—0.5 cm.

Width.—Near 0.2 cm.

Texture/appearance.—Pubescent.

Peduncles: Peduncle length is shortest at the terminal flower and progressively longer moving down the stem; longest length is on average 12 cm; shortest length on average 8 cm.

Length.—9 cm.

Colour.—Near 146 B.

Surface.—Pubescent.

Habit.—Angular and incurving.

Strength.—Medium.

Pedicel:

Length.—Terminal: Near 4 cm; Lateral: Near 6 cm. Colour: Near 146 B.

Surface.—Pubescent.

Habit.—Slightly undulating.

Strength.—Medium.

Ray Florets:

Form/shape.—Straight and slightly incurved.

Texture/appearance.—Matte.

Number per flower.—Approx. 200

Length.—1½ to 2.0 cm; Width: 0.5 cm.

Apex.—Pointed.

Base.—Tapered.

Margin.—Entire.

Disc florets:

Form/shape.—Short and tubular.

Texture/appearance.—Shiny.

Number per flower.—Approx. 20.

Length.—0.3 cm; Width: 0.05 cm.

Diameter of disc.—0.5 cm.

Base.—Tapered.

Fragrance: Slight

Flower Bud (at onset of colour):

Length.—Near 0.7 cm.

Diameter.—Near 1.0 cm.

Form/shape.—Spherical.

General flower colour: Orange bronze.

1. Ray florets, upper side.—Immature: Near 163 A Mature: Near 163 B Older/Fading: Near 163 C

2. Ray florets, under side.—Immature: Near 11 A Mature: Near 11 A Older/Fading: Near 11 B.

3. Disc florets.—Immature: Near 145 A Mature: Near 13 A. Older/Fading: Near 12 B

4. Bud.—Near 138 A.

Flower progression: Flower incurved but outer floret reflexed and slightly faded during ageing.

Other Distinguishing Characteristics: None

### Reproductive Organs

Ray Florets: Pistillate

Pistil number.—100.

Stigma colour.—Near 6 C.

Stigma shape.—Forked.

Style colour.—Near 6 C.

Style length.—Near 0.5 cm.

Disc Florets: Pistillate

Pistil number.—20 to 30.

Stigma colour.—Near 6 C.

Stigma shape.—Forked.

Style colour.—Near 6 C.

Style length.—Near 0.1 cm.

Staminate.—Not Observed.

What is claimed is:

1. A new and distinct variety of Chrysanthemum plant, substantially as described and illustrated herein.
